(19) United States
(12) Patent Application Publication
(10) Pub. No.: US 2003/0022166 A1
(43) Pub. Date: Jan. 30, 2003

(54) Repeat-free probes for molecular cytogenetics

(76) Inventors: Colin Collins, San Rafael, CA (US); Stanislav Volik, Albany, CA (US); Joe W. Gray, San Francisco, CA (US); Donna G. Albertson, Lafayette, CA (US); Daniel Pinkel, Walnut Creek, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/766,450

(22) Filed: Jan. 19, 2001

(51) Int. Cl. C12Q 1/68; G06F 19/00; G01N 33/48; G01N 33/50; C07H 21/04

(52) U.S. Cl. 435/6; 702/20; 536/24.3

(57) ABSTRACT

The present invention provides a rapid, efficient, and automated method for identifying unique sequences within the genome. This invention involves the identification of repeat sequence-free subregions within a genomic region of interest as well as the determination of which of those repeat sequence-free subregions are truly unique within the genome. Once the truly unique subregions are identified, primer sequences are generated that are suitable for the amplification of sequences, e.g., for use as probes or array targets, within the unique subregions.

### STATEMENT AS TO RIGHTS TO INVENTIONS MADE UNDER FEDERALLY SPONSORED RESEARCH AND DEVELOPMENT

This invention was made with Government support under Grant No. CA58207, awarded by the National Institutes of Health. The Government has certain rights in this invention.

### BACKGROUND OF THE INVENTION

Fluorescence in situ hybridization (FISH) and array CGH are powerful techniques that allow the detection of any of a number of genomic rearrangements within a genome, such as a tumor genome (see, e.g., Gray & Collins (2000) Carcinogenesis 21:443-452). In FISH, labeled probes are hybridized to chromosomes, e.g., metaphase chromosomes, thereby allowing the detection of the chromosomal position, copy number, presence, etc. of a specific target sequence in vivo (see, e.g., Speicher et al. (1996) Nature Med. 2:1046-1048; Lichter (1997) Trends Genet. 13:475-479; Raap (1998) Mutat. Res. 400:287-298). Array CGH involves the hybridization of labeled DNA, e.g., genomic DNA, from a plurality of sources to an arrayed set of target sequences. In array CGH, differences in the extent of hybridization (e.g., as measured by fluorescence intensity when fluorescently-labeled genomic DNA is used) of a test genome to a control genome indicate the presence of an alteration, e.g., a change in copy number, in the test genome relative to the control genome (see, e.g., James (1999) J. Pathol. 187:385-395).

FISH, array CGH, and many other hybridization-based methods often depend upon the use of probes or target sequences that include repeat sequences that are found at multiple locations in the genome. The presence of repeat sequences within probes or CGH targets has typically led to the requirement for suppression of the hybridization of the repeated sequences in order to achieve locus specific analysis. This is typically accomplished by including excess unlabeled repeat rich DNA during the hybridization process. While effective, this slows the reaction and often cannot be accomplished completely. In addition, even when hybridization of known repeat sequences is suppressed, the remaining sequences are often not truly unique, but instead have multiple close homologs elsewhere in the genome. For example, various members of a single gene family may be highly homologous yet present in disparate locations in the genome. Probes specific for any one member of the family, therefore, may specifically hybridize to multiple sites within the genome under certain conditions, thereby confounding analysis.

Another problem is high-throughput identification of genes in genomic sequence. Current methods of gene identification are based on combination of two approaches—search of the existing databases of expressed sequences (which may be incomplete) and ab initio prediction of gene structure using programs like Xgrail and Genscan (which do not work efficiently on all genomic sequences). Additionally, after the computer analysis is complete, there is no generally accepted high-throughput and efficient approach for experimental verification of the results of computer analysis.

### SUMMARY OF THE INVENTION

The present invention provides a rapid, efficient, and automated method for identifying unique sequences within the genome. This invention involves the identification of repeat sequence-free subregions within a genomic region of interest as well as the determination of which of those repeat sequence-free subregions are truly unique within the genome. Once the truly unique subregions are identified, primer sequences are generated that are suitable for the amplification of sequences, e.g., for use as probes or array targets, within the unique subregions.

One of the ways of achieving high-throughput identification of genes in a genomic sequence is to utilize the fact that vast majority of genes are encoded in unique part of genomic DNA (or in parts of very low copy number). Thus, after identification of truly unique sequences, one can print them on arrays and use as hybridization targets for mRNA probes (a la expression arrays). This approach is inherently high-throughput and easy to automate, and is independent of any bias towards previously identified expressed sequences. According to another aspect of the present invention, unique, repeat-free probes are produced to provide a convenient method for production of, e.g., probes for FISH, or array targets, which represent truly unique sequences within the genome.

As such, in one aspect, the present invention provides a method for identifying oligonucleotide sequences suitable for the amplification of a unique sequence within a genomic region of interest, the method comprising the steps of (i) executing a first process to identify repeat sequences that occur within the genomic region of interest; (ii) executing a second process to compare repeat sequence-free subsequences within the genomic region of interest to a nucleotide sequence database, whereby nucleotide sequences within the nucleotide sequence database that are substantially similar to the repeat sequence-free subsequences are identified; (iii) executing a third process to identify oligonucleotide sequences that are suitable for use as primers in an amplification reaction to amplify a product within any of the repeat sequence-free subsequences for which a defined number of substantially similar sequences are identified in said nucleotide sequence database; and (iv) outputting the oligonucleotide sequences.

In one embodiment, the genomic region is from a human genome. In another embodiment, the defined number of substantially similar sequences is zero. In another embodiment, the sequences are outputted by displaying the sequences on a computer screen or on a computer printout. In another embodiment, the sequences are outputted by executing a fourth process on a digital computer to direct the synthesis of oligonucleotide primers comprising the oligonucleotide sequences. In another embodiment, the computer directs the synthesis of the oligonucleotide primers by ordering the synthesis from an external source, such as a commercial supplier. In another embodiment, the computer is in communication with an oligonucleotide synthesizer, and the synthesis is performed by the synthesizer. In another embodiment, the substantially similar sequences are at least about 50% identical to the repeat sequence-free subsequences. In another embodiment, the substantially similar sequences are at least about 70% identical to the repeat-sequence free subsequences. In another embodiment, the substantially similar sequences are at least about 90% identical to the repeat-sequence free subsequences. In another embodiment, the first process is executed using Repeat Masker software. In another embodiment, the second process is executed using a BLAST algorithm. In another embodiment, the third process is executed using Primer3 software. In another embodiment, the method further comprises generating an amplification product using the oligonucleotide primers. In another embodiment, the amplification product is a FISH probe. In another embodiment, the FISH probe is fluorescently labeled. In another embodiment, the amplification product is an array CGH target. In another embodiment the amplification product is an array target for hybridization with labeled mRNA of interest. In another aspect, the present invention provides a method for visually displaying oligonucleotide sequences suitable for the amplification of a unique sequence within a genomic region of interest, the method comprising the steps of (i) analyzing a genomic nucleotide sequence that encompasses the genomic region of interest to identify repeat sequences within the genomic region; (ii) comparing at least one repeat sequence-free subsequence within the genomic nucleotide sequence to a nucleotide sequence database to identify sequences within the database that are substantially similar to the repeat sequence-free subsequence; (iii) for at least one of the repeat sequence-free subsequences for which a defined number of substantially similar sequences are identified within the nucleotide sequence database, selecting oligonucleotide sequences that are suitable for use as primers in an amplification reaction to amplify a product within the repeat sequence-free subsequence; and (iv) displaying the oligonucleotide sequences.

In one embodiment, the genomic region is from a human genome. In another embodiment, the defined number of substantially similar sequences is zero. In another embodiment, the substantially similar sequences are at least about 50% identical to the repeat sequence-free subsequences. In another embodiment, the substantially similar sequences are at least about 70% identical to the repeat sequence-free subsequences. In another embodiment, the substantially similar sequences are at least about 90% identical to the repeat sequence-free subsequences. In another embodiment, the identification of repeat sequences within the genomic region is performed using Repeat Masker software. In another embodiment, the comparison of the at least one repeat sequence-free subsequence with the genome database is performed using a BLAST algorithm. In another embodiment, the oligonucleotide sequences are selected using Primer3 software.

In another aspect, the present invention provides a computer program product visualizing oligonucleotide sequences suitable for use as primers to amplify unique sequences within a genomic region of interest, the computer program product comprising a storage structure having computer program code embodied therein, the computer program code comprising (i) computer program code for causing a computer to analyze a nucleotide sequence encompassing the genomic region of interest to identify repeat sequences within the nucleotide sequence; (ii) computer program code for causing a computer to, for each subsequence of the nucleotide sequence that does not contain any of the repeat sequences, compare the subsequence against a nucleotide sequence database to identify nucleotide sequences within the database that are substantially similar to the subsequence; (iii) computer program code for causing a computer to, for each of the subsequences for which a defined number of substantially similar sequences are found in the database, identify oligonucleotide sequences suitable for use as primers in an amplification reaction to amplify a product within the subsequence; and (iv) computer program code for displaying the oligonucleotide sequences.

In one embodiment, the defined number of substantially similar sequences is zero. In another embodiment, the substantially similar sequences are at least about 50% identical to the subsequences. In another embodiment, the substantially similar sequences are at least about 70% identical to the subsequences. In another embodiment, the substantially similar sequences are at least about 90% identical to the subsequences.

### BRIEF DESCRIPTION OF THE DRAWINGS

FIG. 1 provides a flow chart of the basic steps involved in the present invention. To identify unique sequences within the region of interest, known repeat sequences (“R”) are removed, e.g., using a program such as Repeat Masker. The remaining, repeat sequence-free subsequences (“A,” “X,” “D” and “Y”) are searched against a genomic database to identify potential homologs located elsewhere in the genome. Subsequences with homologous sequences elsewhere in the genome (“A,” “D”) are discarded, and primer sequences are designed that are suitable for the amplification of the remaining, unique sequences (“X,” “Y”).

FIG. 2 provides a flow chart showing a preferred embodiment of the computational steps used to practice the invention. A “sequence,” corresponding to, e.g., a genomic region of interest, is analyzed using Repeat Masker to identify known repeat sequences within the sequence. The identified repeat sequences are both displayed and removed from the “sequence,” providing a “masked sequence.” The masked sequence is then used to perform BLAST searches against one or more genomic databases, and then unique sequences within the masked sequence are selected. Primer sequences are then designed based on the selected unique sequences, and are displayed along with supplemental information such as the PCR conditions, the cost of the primers, etc. The names of programs from public domain are shown in italics. The final output is presented in pentagrams. Intermediate data are shown in rectangles. The input information input into the major module (unique_DNA.pl) is shown by feathered arrows.

### DESCRIPTION OF THE SPECIFIC EMBODIMENTS

I. Introduction

The present invention provides a novel and efficient method for identifying unique sequences within the genome. This method involves the use of computational analysis to identify sequences anywhere within a genome that are homologous to the locus to be tested. This is now feasible because of the availability of complete genomic sequence of most or all of the human and other genomes. In a typical embodiment, once the locations of the repeated regions are known, PCR primers are designed to amplify most or all of the remaining unique sequences. The PCR fragments can then be labeled and used as FISH probes or printed as DNA array elements. Alternatively, the PCR fragments can be cloned into plasmid or other vectors and the clones can be propagated to produce FISH probes or array targets. Either method allows FISH or array hybridization to be carried out without including blocking DNA during the hybridization process, thereby increasing the speed and specificity of the reaction.

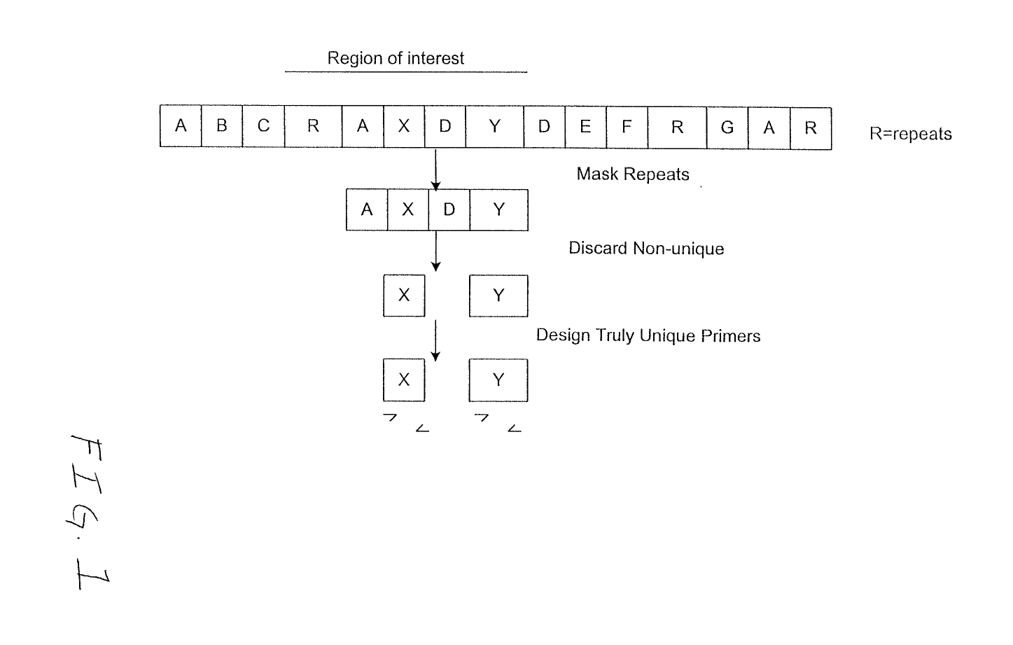

In a preferred embodiment, the present invention involves several computer-based steps for identifying unique sequences within a genomic region of interest. As depicted in FIG. 1, the first of these steps involves the removal of repetitive sequences from a sequence corresponding to the genomic region. Once the repetitive sequences are removed, the remaining large sequences are used to search one or more databases of genomic sequences to identify the sequences that are truly unique within the genome (or which have a defined number of close homologs), i.e., non-unique sequences are discarded. Those sequences that are found to lack both known repetitive sequences as well as close homologs elsewhere in the genome are then used to design primers that would allow amplification of unique products for use as probes or array targets.

II. Genomic Sequence

The present methods can be used to identify unique sequences within any genomic region of interest. The genomic region can be any of a large range of sizes, e.g., 1 kb, 10 kb, 100 kb, 1 Mb, 10 Mb, or larger, provided that the region to be analyzed has been sequenced. Typically, the genomic region will correspond to a region for which a probe is desired, e.g., a region rearranged in tumor cells, a region serving as a chromosomal marker for in situ hybridization, etc. In some embodiments, the region will correspond to a genetic interval thought to contain a gene, and the methods are used to identify unique sequences within the interval as a way of identifying coding sequences within the interval.

The genomic region analyzed in this method can be from any genome, so long that a substantial proportion of the genome has been sequenced and is present in an accessible database. Such genomes thus include viral, prokaryotic and eukaryotic genomes, including fungal, plant, and animal genomes, including mammals and, preferably, humans.

III. Removing Repeat Sequences

Typically, the first step of the present methods involves the identification of subregions within the genomic region of interest that lack known repeat sequences. This step can be performed in any of a number of ways, e.g., using any of a number of readily available computer programs. Preferably, the step will involve the identification of repeat sequences within the region, which can then be displayed, as well as the automatic generation of a “masked” sequence from which the repeat sequences have been removed.

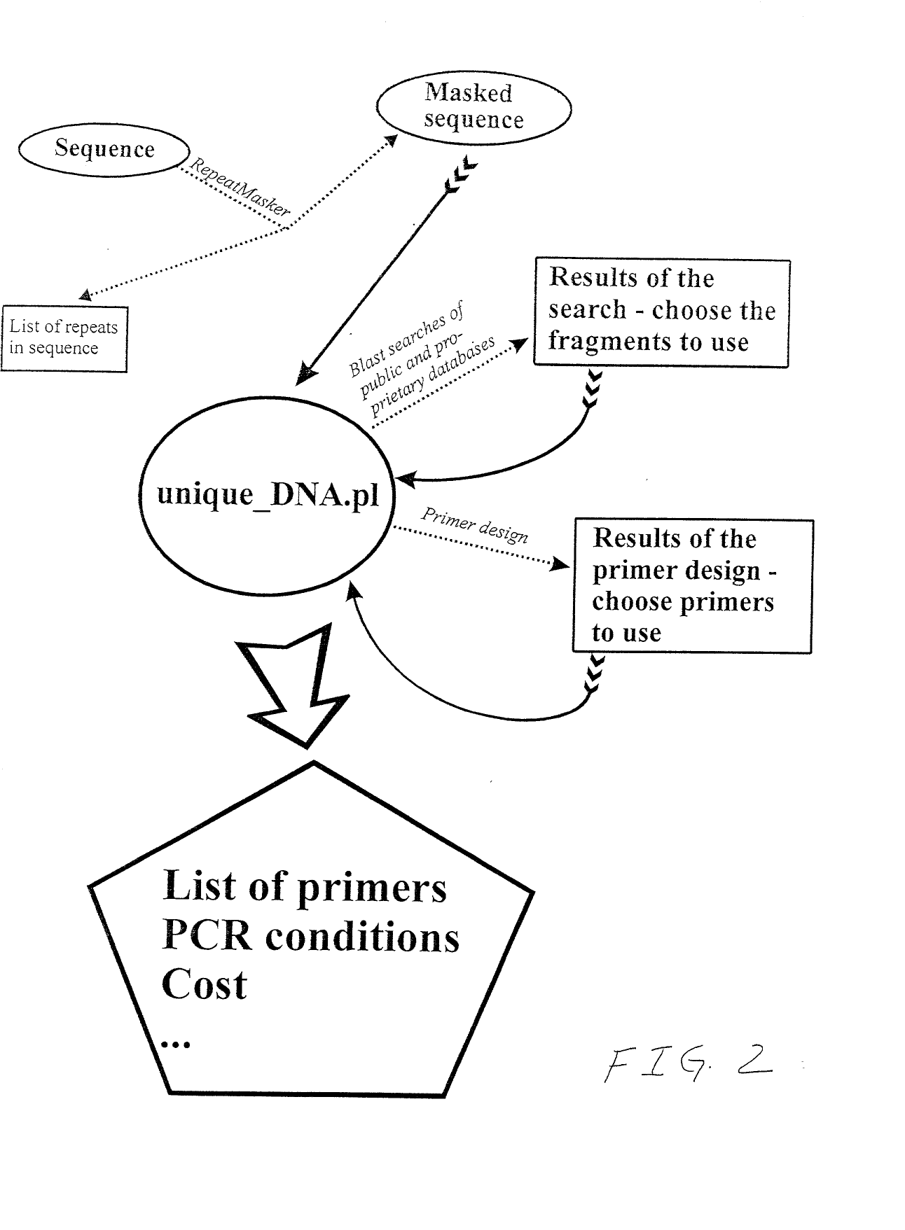

In a preferred embodiment, as depicted in FIG. 2, the process is carried out using any version of the RepeatMasker program (Arian Smit, University of Washington, Seattle, Wash.), such as RepeatMasker2. This program screens sequences for interspersed repeats that are known to exist in mammalian genomes, as well as for low complexity DNA sequences. The output of the program includes a detailed annotation of the repeats present in the query sequence, as well as a modified (“masked”) version of the query sequence in which all the annotated repeats have been masked (e.g., replaced by Ns). The RepeatMasker program is publicly available (see, e.g., http://repeatmasker.genome.washington.edu/).

Other usable programs include Censor (Jurka, et al. (1996) Computers and Chemistry 20:119-122; see, e.g., http://www.girinst.org/Censor_Server.html; Genetic Information Research Institute, California); Satellites or Repeats (Institut Pasteur, Paris; see, e.g., http://bioweb.pasteur.fr/seqanal/interfaces); and others.

IV. Searching Remaining Sequences Against Genome Databases

Once the original DNA sequences has been processed for repeat sequences, e.g., by a program such as RepeatMasker, the coordinates of all of the repeat sequence-free subsequences within the overall sequence are identified from the output file of the program and saved. These coordinates are used to generate a visual display of the repeat-free subsequences, e.g., as a histogram or text file that contains the information on the content and size distribution of repeat-free DNA, including such information as the percentage of the starting sequence that is contained in the subsequences of any given length. In this way, the user can select a suitable threshold for the size of the subsequences to be analyzed in subsequent steps. Once selected, all of the remaining subsequences that are larger than the selected (or preprogrammed) threshold are extracted and saved to files. The size threshold can be essentially any size, e.g., 100 bp, 500 bp, 1 kb, or greater. The following tables are examples of the above described histograms:

| Interval | Number of | Number of |
|---|---|---|
| range | fragments | bases |
| An example of unique frequent size distribution: | | |
| <100 | 83 | 2184 |
| 100-200 | 25 | 3547 |
| 200-300 | 25 | 5904 |
| 300-400 | 12 | 4101 |
| 400-500 | 9 | 4155 |
| 500-600 | 9 | 4935 |
| 600-700 | 9 | 6035 |
| 700-800 | 4 | 3031 |
| 800-900 | 5 | 4356 |
| 900-1000 | 6 | 5711 |
| >1000 | 14 | 21324 |
| Total number of unique bases- | | 65283 |
| And on BAC 189 (649293-784927) | | |
| <100 | 258 | 5214 |
| 100-200 | 50 | 7436 |
| 200-300 | 31 | 7808 |
| 300-400 | 18 | 6109 |
| 400-500 | 13 | 5922 |
| 500-600 | 3 | 1589 |
| 600-700 | 4 | 2624 |
| 700-800 | 3 | 2264 |
| 800-900 | 3 | 2504 |
| 900-1000 | 2 | 1901 |
| >1000 | 9 | 15047 |

| | |
|---|---|
| Total number of unique bases- | 58418 |

The selected subsequences are then searched against one or more genomic databases to identify homologous sequences located elsewhere in the genome. The genome database can be any database that contains a significant amount of sequence information from the same organism as the genomic region being analyzed. While the database preferably contains the entire genomic sequence of the organism, incomplete databases can also be used, allowing the generation of nearly unique sequences that are still useful for a number of applications.

Examples of suitable databases include GenBank, ACEDB (A Caenorhabditis elegans DataBase), the Bacillus Subtilis Genetic Database, Bean Genes (a plant genome database which contains information relevant to Phaseolus and Vigna species), ChickBASE (a database of the chicken genome), FlyBase, GSDB (Genome Sequence Data Base), GrainGenes (a USDA-sponsored database providing molecular and phenotypic information on wheat, barley, rye, oats, and sugarcane), Influenza Sequence Database (contains sequence database and analysis tools regarding influenza A, B, and C viruses), the Japan Animal Genome Database, the Malaria Database, the Methanococcus jannaschii Genome Database, the Mosquito Genomics WWW Server, the RATMAP (the Rat Genome Database), the Saccharomyces Genome Database, the SoyBase (a USDA soybean genome database), the STD Sequence Databases (contains genomic databases of Chlamydia trachomatis, Mycoplasma genitalium, Treponema pallidum, and Human Papillomavirus), the Arabidopsis Information Resource (TAIR), the TIGR Database (TDB), or any other genomic database.

Typically, the masked sequence (i.e., collection of selected subsequences) will be compared with the genome database using a suitable algorithm such as BLAST (see, e.g., the BLAST server at the National Center for Biotechnology Information; http://www.ncbi.nlm.nih.gov/). A BLAST or equivalent search will identify sequences within the genome that are homologous to the masked sequence, preferably ranked in order of similarity to each subsequence.

For sequence comparison, typically one sequence (e.g., a particular repeat sequence-free subsequence) acts as a reference sequence, to which test sequences (e.g., sequences from the genome database) are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are preferably used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The result of these database searches will be a set of sequences, preferably ranked according to percent identity, that are homologous to each of the subsequences. In many embodiments, each of the subsequences that have any close homologs (e.g., with a percent identity of greater than 50%, 60%, 70%, 80%, 90%, 90% or higher) elsewhere in the genome will be discarded. The particular degree of homology of the sequence that will warrant removal will depend on any of a large number of factors, including the particular application the probes or target sequences will be used for, the hybridization conditions that will be used, the number of homologs identified (for the particular subsequences as well as for other subsequences within a given genetic interval), the total number of potential subsequences, the need for absolute uniqueness of a probe, etc.

In numerous embodiments, repeat sequence-free subsequences that have a limited number of close homologs will be deliberately selected, as such sequences might represent members of a gene family. Accordingly, primers specific to that subsequence, or probes generated using the primers, may be useful in the identification of other members of the same family. Accordingly, in certain embodiments, the user will be able to select the number of close homologs (e.g., 0, 1, up to 2, up to 5, etc.) that a selected subsequence may have.

V. Designing Primer Sequences

Once one or more particular subsequences are selected, primers are designed that are suitable for the amplification of one or more of the subsequences, or portions thereof. The primers can be designed to amplify a product of any size, e.g., 100 bp, 1 kb, 5 kb, 10 kb, 50 kb, or larger; the size of the desired product is a parameter than can be selected for particular applications.

Typically, the primers will be designed not only based on the size of the product, but also taking into account any of a large number of considerations for optimal primer design, e.g., to exclude potential secondary structures within the primers, with a desired Tₘ (that is preferably similar for each member of a pair of primers), to include additional sequences such as restriction sites to facilitate cloning of the amplified product, etc. Examples of suitable programs for designing (and analyzing potential primer sequences) include, but are not limited to, Primer3 (from the Whitehead Institute; http://www.genome.wi.mit.edu/cgi-bin/primer/primer3.cgi), PrimerDesign (http://www.chemie.uni-marburg.de/˜becker/pdhome.html), Primer Express® Oligo Design Software (PE Biosystems), DOPE2 (D)esign of Oligonucleotide Primers; http://dope.interactiva.de/); DoPrimer (http://doprimer.interactiva.de); NetPrimer (http://www.premierbiosoft.com/netprimer.html); Oligos-U-Like—Primers3 (http://www.path.cam.ac.uk/cgi-bin/primer3.cgi); Oligo (v5.0); CpG Ware™ Primer Design Software, PrimerCheck (http://www.chemie.uni-marburg.de/becker/freeware/freeware.html#primercheck), and others. General parameters for designing primers can be found in any of a large number of resources and publications, including Dieffenbach, et al., in PCR Primer, A Laboratory Manual, Dieffenbach et al., Ed., Cold Spring Harbor Laboratory Press, New York (1995), pp. 133-155; Innis, et al., in PCR protocols, A Guide to Methods and Applications, Innis, et al., Ed., CRC Press, London (1994), pp. 5-11; Sharrocks, in PCR Technology Current Innovations, Griffin, H. G., and Griffin, A. M, Ed., CRC Press, London (1994) 5-11.

VI. Displaying Primer Sequences and Other Information

Once suitable primer sequences have been designed, they are preferably displayed, in any readable format, preferably along with information regarding the primers, reaction conditions, etc. Examples of information that can be displayed along with the primer sequences include, but is not limited to, the size of the primers, the size of the anticipated amplified product, the melting temperature of the primers, the G/C content of the primers, restriction sites or any other functional entities encoded in the primers, the genomic localization of the predicted amplified sequences, the cost of primer synthesis, and suitable reaction conditions for various reactions (e.g., PCR) including the primers. The following is an example of a primer file:

```
675342.f1 TGCATCTGGGAGGGTGTC
675342.r1 AACCAATCCCAAGGATCCAG
  TmL = 60.65; TmR = 61.08; product size = 1002
673920.f1 GACCTCACTGCTCCTGAACC
673920.r1 TCTGCAACCTTTGCTTTCTG
  TmL = 59.84; TmR = 59.19; product size = 998
759724.f1 CAACATTTGGTTGCAGTCATC
759724.r1 TGTGTCTTTTTCTTCCCTCAAAG
  TmL = 59.04; TmR = 59.79; product size = 996
652197.f1 GGAGCATGCAAAAGAGGATG
652197.r1 CAGATCCCACTGCCATTAGC
  TmL = 60.74; TmR = 60.62; product size = 1185
746914.f1 GGAGTAAAGGAGGCTGACTGG
746914.r1 CACCACAGCAGTAAGCTGAAAG
  TmL = 60.25; TmR = 60.11; product size = 1333
770028.f1 TTTTCAGAGGCTTCCCATAGTC
770028.r1 TGCTTTTCCATTCCTGCTTC
  TmL = 59.73; TmR = 60.33; product size = 1277
748329.1.f1 AAAGCATAGGAAACATCCAAATG
748329.1.r1 TCGATCAAGCTTTCAAAGGAC
  TmL = 59.41; TmR = 59.44; product size = 829
748329.2.f1 AACCCGGGAGGTTGTCAG
748329.2.r1 TTTGCATGTTTTGCATTTGG
  TmL = 60.92; TmR = 60.49; product size = 808
656003.1.f1 TTGAATTTTTCATCGGTCAGG
656003.1.r1 CCCTGGATTTCAGCTGTTTC
  TmL = 59.92; TmR = 59.67; product size = 967
656003.2.f1 ATCACCTTCATTCCCTCTGG
656003.2.r1 TGACCACATTTCTGCCTTTG
  TmL = 58.94; TmR = 59.69; product size = 985
650954.f1 GAACGCAGCTTTCCTTTTTG
650954.r1 GGGAAGACAACTCTTGGAAATG
  TmL = 60.00; TmR = 59.98; product size = 211
654685.f1 GCAACTTTCTCCGGGTTAGAG
654685.r1 CAGCTGTGTACTGTTTGGCTTG
  TmL = 60.25; TmR = 60.91; product size = 229
663047.f1 AGGGAAGAGAGGTGTCTCAGC
663047.r1 AAAAAGCCAGTGCTTTCTGG
  TmL = 60.01; TmR = 59.49; product size = 274
683270.f1 AACTGTGGGGCCTTTAGATG
683270.r1 CAGGGTTTTCCCACAGAAAG
  TmL = 59.05; TmR = 59.56; product size = 268
683663.f1 GGACAAGCTGGTTTCCTTTC
683663.r1 AATATTTACAGCGCCTGTTGC
  TmL = 58.77; TmR = 59.29; product size = 232
695950.f1 GTAAAGCCCCTGACATCCAG
695950.r1 AACTTCCCAACAGCCAAGC
  TmL = 59.55; TmR = 60.25; product size = 261
711254.f1 AAACGCTCCATTGCTGCTAC
711254.r1 GCCAGACTGGGATCTACCTG
  TmL = 60.42; TmR = 59.68; product size = 240
716931.f1 ATGTCTCTGGGCATCTGGAG
716931.r1 TTGGAAAAACAAATTGTACCTCAC
  TmL = 60.22; TmR = 59.35; product size = 300
723983.f1 AACCCCAATTTTGTTTCAAGTG
723983.r1 ATTCCAAAATGCCTGACTGC
  TmL = 60.12; TmR = 60.08; product size = 355
727725.f1 AGTTCCAGCAGGGAGGAATC
727725.r1 GTGTCGATGGTTTTTACAAGAGG
  TmL = 60.60; TmR = 59.92; product size = 274
732837.f1 CTGATTCAGAAGCTGGACTGG
732837.r1 AGCATTTGGCTGTGTGACC
  TmL = 60.00; TmR = 59.70; product size = 365
738261.f1 TGATGCTGACCAGGAAAAAC
738261.r1 AGCTGATGAGGCAGAAAAGG
  TmL = 58.70; TmR = 59.57; product size = 208
756209.f1 TCTAAAAATGGGGCACAAGG
756209.r1 CTTCCCTTGCCCCTAACAG
  TmL = 59.93; TmR = 59.67; product size = 337
768348.f1 TTTTCTGGTTGCAGGATTGG
768348.r1 AACACATGCACACGCACAC
  TmL = 61.00; TmR = 60.24; product size = 282
777535.f1 GAAAGGAAAAATATCCCAGAGG
777535.r1 AAATGCTGGCCTTATTTTCAC
  TmL = 58.15; TmR = 58.26; product size = 241
783903.f1 GCAGCTGAAAACTTAACCCAAG
783903.r1 AATGCAGAGAATGAAGACTGAATG
  TmL = 60.29; TmR = 59.79; product size = 207
733241.1.f1 CCAGGACCTGCCTCTCAG
733241.1.r1 TGCCTGTCTGCTGTTTTCTG
  TmL = 59.47; TmR = 60.18; product size = 1314
733241.2.f1 TGGGAGTCACTCAAGTGCAG
733241.2.r1 AATTCGATCCATTTTTCTTTGG
  TmL = 60.02; TmR = 59.34; product size = 1262
733241.3.f1 GCCCTTTCCTGTGGTTTTTAG
733241.3.r1 GGGAGAGAGAAAAGGACAACG
  TmL = 59.99; TmR = 60.23; product size = 1306
660316.f1 CACTTCAAATCTTGAAAAGTTCTGG
660316.r1 CAGACTGCATTGGCCTGAG
  TmL = 60.52; TmR = 60.56; product size = 396
672598.f1 TCTGCAATTTTTAACCATTTATGAG
672598.r1 CTTTTCCAGGGGGAAATACAC
  TmL = 58.73; TmR = 59.69; product size = 457
676658.f1 GCAAAGGGACACGTCTAGGT
676658.r1 CTGTTTTCGACACAACACCAA
  TmL = 59.21; TmR = 59.64; product size = 341
681855.f1 CCAGCTGTGCAGATTTCTTTC
681855.r1 ATTCAGCAGCCCATGGTTAC
  TmL = 60.01; TmR = 59.96; product size = 441
687779.f1 TCCTGAAGATGCTGAGTCAATG
687779.r1 GGCTGCAGTAGGTTCCAAAG
  TmL = 60.40; TmR = 59.88; product size = 390
719646.f1 ACAAGGGTGCAGGTGAAAAC
719646.r1 AATAGCCAACACCACCTTCTTC
  TmL = 60.01; TmR = 59.53; product size = 395
730564.f1 CCTCAGGGAAGATCAGACTCC
730564.r1 TTTGTGAAACTTTTTGCTGTGTG
  TmL = 60.20; TmR = 60.23; product size = 414
745381.f1 TCGCAGATCAAGGCTTACAG
745381.r1 TGTGGTGAAAAACCAATACTGC
  TmL = 59.17; TmR = 59.90; product size = 428
750823.f1 GAACCAGGCCAGAGTTTTTG
750823.r1 ATGTGGGGCATGTGACTTC
  TmL = 59.71; TmR = 59.33; product size = 386
753539.f1 TAAACCCAGGCTCAGCAATG
753539.r1 AAAATGCTGCCCTTCCTTTC
  TmL = 61.16; TmR = 60.56; product size = 368
762267.f1 GGACGTTCATTTGGATTTGC
762267.r1 GGGTGCCGTTCCATTTATTAG
  TmL = 60.32; TmR = 60.55; product size = 369
767583.f1 CCACTCTGCCATAGCACTTC
767583.r1 AAAGCCCCATTATGAACTCG
  TmL = 58.47; TmR = 59.04; product size = 414
775788.f1 TGCCCATATGCTATTGTATCTGTC
775788.r1 TCCTCTCATCCCAGTTCCTG
  TmL = 60.25; TmR = 60.19; product size = 297
692036.f1 GTGTGTGAATGGCAGGTTTG
692036.r1 GGGGGCAGTTACCAAAAGAC
  TmL = 60.01; TmR = 60.72; product size = 476
707612.f1 GCATCTGGTTGCCTTACCTC
707612.r1 CGCATGTATCAGGAATGAAGC
  TmL = 59.70; TmR = 60.62; product size = 480
709543.f1 CCCCAAATGGGATAAAGAGG
709543.r1 AGAGGGAAAAACGTGAAGGAG
  TmL = 60.49; TmR = 59.74; product size = 494
714041.f1 CTCCACTGAATTTTCCCATTC
714041.r1 TCCAAGTGAAATGAAAAACTGG
  TmL = 58.49; TmR = 59.11; product size = 578
764904.f1 GGAGCCTCTTTTCATTATACAGC
764904.r1 GATTTAACAAGGGCAAAAGAGC
  TmL = 58.50; TmR = 59.29; product size = 650
773843.f1 TCAGCAGGTGAACAGCACAG
773843.r1 ATGGGTGATCAAACCACAGC
  TmL = 61.24; TmR = 60.79; product size = 550
781783.f1 AAGCAGGGGCACTGAATATG
781783.r1 CAGAGCTGGGTTTGGTAAGC
  TmL = 60.10; TmR = 59.88; product size = 558
703668.f1 AGTGACTCCCTGCTGTGAAAG
703668.r1 AAGCTGTGATTCCGTTCCAC
  TmL = 59.51; TmR = 60.12; product size = 756
744236.f1 CCTGCAGGAAGGGTGTATTC
744236.r1 TCTCTGAACAGCAGTCATAGCAC
  TmL = 59.55; TmR = 59.70; product size = 626
651312.f1 GCACCTCCAGAAGGGAGAG
651312.r1 TGTGGCAAATTCAAGACCAG
  TmL = 59.93; TmR = 59.69; product size = 758
731993.f1 AGCCCCAAACCTTCAAGC
731993.r1 TCCACCTATTTTTCAACACACG
  TmL = 60.20; TmR 59.90; product size = 768
752055.f1 TTCCTAAGTTTAACCCCACAGG
752055.r1 CAAAACCATTAGGTGGAGAGC
  TmL = 59.41; TmR = 58.71; product size = 757
653556.f1 TTTCTCCATGAACAAATAGGAATG
653556.r1 AACTGGGAACCGCATAATTG
  TmL = 59.39; TmR = 59.82; product size = 771
702011.f1 CACTGAAGCCAAAATAAGTTCC
702011.r1 CAGAGTGCCACTGGTCTAGG
  TmL = 57.94; TmR = 58.46; product size = 922
Total number of bases to be ordered—2322
Total length of PCR products—32786
```

Because a plurality of suitable primer pairs will likely be available for any given genomic region, the present process can be programmed to design primers for all suitable subregions within the region, or to automatically select one or more suitable primer pairs, for example based on various parameters that can be preselected by the user, to generate a small, optionally predetermined number of probes. Alternatively, a number of possible primers can be displayed, along with information about their use, cost, product, etc., and one or more particular sets can be selected by the user.

VII. Synthesize/Order the Primers

Once a suitable primer set has been selected, either manually or automatically as described supra, the program can automatically order the synthesis of the primers, e.g., from any of a large number of commercial suppliers of oligonucleotides. Alternatively, if available, the program can also direct the synthesis of primers having the selected sequences using local facilities in communication with a computer running the program. When the primers are ordered or synthesized, they are preferably displayed along with the date of ordering, the particular supplier, the expected date of delivery, etc.

It will be appreciated that the primers can be made using any method (e.g., the solid phase phosphoramidite triester method described by Beaucage and Caruthers (1981), Tetrahedron Letts., 22(20):1859-1862, using an automated synthesizer, as described in Needham-VanDevanter et al. (1984) Nucleic Acids Res., 12:6159-6168), and including any naturally occurring nucleotide or nucleotide analog and/or inter-nucleotide linkages, all of which are well known to those of skill in the art. Examples of such analogs include, without limitation, phosphorothioates, phosphoramidates, methyl phosphonates, chiral-methyl phosphonates, 2-O-methyl ribonucleotides, peptide-nucleic acids (PNAs). The use of labeled nucleotides, e.g., fluorescent nucleotides, in the preparation of primers is also contemplated.

VIII. Using Primers to Generate Unique Probes

The unique sequences provided by the present invention can be used for any of a large number of applications. In a preferred embodiment, the sequences are used to make probes for applications such as FISH or array targets (for array CGH or hybridization with labeled mRNA of interest). In such embodiments, the probes or array targets can be used without adding an excess of additional unlabeled repeat sequences, thereby enhancing the speed, simplicity, and efficiency of the reaction compared to traditional methods.

To generate the probes, the synthesized primers are typically used in an amplification reaction such as PCR to amplify the unique sequences, using appropriate sources of template DNA. Template DNA can be derived from any source that includes the region to be amplified, including genomic DNA and cloned DNA (e.g., in a BAC, YAC, PAC, etc., vector). Cloned template DNA can represent a complete or partial library, or can represent a single clone that includes the subsequence of interest.

PCR or any other hybridization reaction using the primers can be performed using any standard method, as taught in any of a number of sources. See, e.g., Innis, et al., PCR Protocols, A Guide to Methods and Applications (Academic Press, Inc.; 1990, Sambrook et al. (1989) Molecular Cloning, A Laboratory Manual (2d Edition), Cold Spring Harbor Press, Cold Spring Harbor, N.Y.; Ausubel et al., eds. (1996) Current Protocols in Molecular Biology, Current Protocols, a joint venture between Greene Publishing Associates, Inc. and John Wiley & Sons, Inc.; Mullis et al., (1987) U.S. Pat. No. 4,683,202, and Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564.

In many embodiments, the unique amplification products will be labeled during the amplification reaction, for example to enable their use in FISH. For example, fluorescently labeled nucleotides, which are well known to those of skill in the art and which are available from any of a large number of sources, can be included. Other nucleotide analogs include nucleotides with bromo-, iodo-, or other modifying groups, which groups affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them. Such modified nucleotides are well known in the art and are available from any of a large number of sources, including Molecular Probes (Eugene, Oreg.); Enzo Biochem, Inc.; Stratagene, Amersham, PE Biosystems, and others.

Because the unique sequences likely represent genes, the present methods are also useful for the identification of candidate genes within a genetic interval, e.g., a genetic interval known to contain a disease-causing gene. In such embodiments, the methods are thus used as a way to identify potential coding sequences within the region. In preferred embodiments, the unique sequence-specific primers are used to amplify sequences from, e.g., a cDNA library generated from cells likely to express the disease-causing gene (such as from a cell type or tissue directly affected by the disease). In this way, coding sequences that are expressed in a particular cell type, and which are expressed from genes lying within a given genetic interval, can be easily identified. These coding sequences represent strong candidates for the disease causing gene.

In a preferred embodiment, the acts described above are performed by a digital computer executing program code stored on a computer readable medium. The program code may be stored, for example, in magnetic media, CD, optical media, or as digital information encoded on an electromagnetic signal.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications and patent documents cited in this application are incorporated by reference in their entirety for all purposes to the same extent as if each individual publication or patent document were so individually denoted.

What is claimed is:

1. A method for identifying oligonucleotide sequences suitable for the amplification of a unique sequence within a genomic region of interest, said method comprising the steps of:
executing a first process on a digital computer to identify repeat sequences that occur within said genomic region of interest;
executing a second process on a digital computer to compare repeat sequence-free subsequences within said genomic region of interest to a nucleotide sequence database, whereby nucleotide sequences within said nucleotide sequence database that are substantially similar to said repeat sequence-free subsequences are identified;
executing a third process on a digital computer to identify oligonucleotide sequences that are suitable for use as primers in an amplification reaction to amplify a product within any of said repeat sequence-free subsequences for which a defined number of substantially similar sequences are identified in said nucleotide sequence database; and
outputting said oligonucleotide sequences.

2. The method of claim 1, wherein said genomic region is from a human genome.

3. The method of claim 1, wherein said number of substantially similar sequences is zero.

4. The method of claim 1, wherein said oligonucleotide sequences are outputted by displaying the sequences on a computer screen or on a computer printout.

5. The method of claim 1, wherein said oligonucleotide sequences are outputted by executing a fourth process on a digital computer to direct the synthesis of oligonucleotide primers comprising said oligonucleotide sequences.

6. The method of claim 5, wherein said computer directs the synthesis of said oligonucleotide primers by ordering said synthesis from an external source.

7. The method of claim 5, wherein said computer is in communication with an oligonucleotide synthesizer, and wherein said computer directs the synthesis of said oligonucleotide primers by said synthesizer.

8. The method of claim 1, wherein said substantially similar sequences are at least about 50% identical to said repeat sequence-free subsequences.

9. The method of claim 1, wherein said substantially similar sequences are at least about 70% identical to said repeat sequence-free subsequences.

10. The method of claim 1, wherein said substantially similar sequences are at least about 90% identical to said repeat sequence-free subsequences.

11. The method of claim 1, wherein said first process is executed using Repeat Masker software.

12. The method of claim 1, wherein said second process is executed using a BLAST algorithm.

13. The method of claim 1, wherein said third process is executed using Primer3 software.

14. The method of claim 5, further comprising producing an amplification product using said oligonucleotide primers.

15. The method of claim 14, wherein said amplification product is a FISH probe.

16. The method of claim 15, wherein said FISH probe is fluorescently labeled.

17. The method of claim 14, wherein said amplification product is an array CGH target.

18. A method for identifying oligonucleotide sequences suitable for the amplification of a unique sequence within a genomic region of interest, said method comprising the steps of:
analyzing a genomic nucleotide sequence that encompasses said genomic region of interest to identify repeat sequences within said genomic region;
comparing at least one repeat sequence-free subsequence within said genomic nucleotide sequence to a nucleotide sequence database to identify sequences within said database that are substantially similar to said repeat sequence-free subsequence;
for at least one of said repeat sequence-free subsequences for which a defined number of substantially similar sequences are identified within said nucleotide sequence database, selecting oligonucleotide sequences that are suitable for use as primers in an amplification reaction to amplify a product within said repeat sequence-free subsequence.

19. The method of claim 18, wherein said genomic region is from a human genome.

20. The method of claim 18, wherein said defined number of substantially similar sequences is zero.

21. The method of claim 18, further comprising displaying said oligonucleotide sequences on a computer screen or on a computer printout.

22. The method of claim 18, further comprising directing the synthesis of oligonucleotide primers comprising said oligonucleotide sequences.

23. The method of claim 22, wherein said synthesis is directed by ordering the synthesis of said primers from an external source.

24. The method of claim 18, wherein said substantially similar sequences are at least about 50% identical to said repeat sequence-free subsequences.

25. The method of claim 18, wherein said substantially similar sequences are at least about 70% identical to said repeat sequence-free subsequences.

26. The method of claim 18, wherein said substantially similar sequences are at least about 90% identical to said repeat sequence-free subsequences.

27. The method of claim 18, wherein the identification of repeat sequences within said genomic region is performed using Repeat Masker software.

28. The method of claim 18, wherein the comparison of said at least one repeat sequence-free subsequence with said genome database is performed using a BLAST algorithm.

29. The method of claim 18, wherein said oligonucleotide sequences are selected using Primer3 software.

30. The method of claim 22, further comprising generating an amplification product using said oligonucleotide primers.

31. The method of claim 30, wherein said amplification product is a FISH probe.

32. The method of claim 31, wherein said FISH probe is fluorescently labeled.

33. The method of claim 30, wherein said amplification product is an array CGH target.

34. A computer program product designing and outputting oligonucleotide sequences suitable for use as primers to amplify unique sequences within a genomic region of interest, said computer program product comprising:
a storage structure having computer program code embodied therein, said computer program code comprising:
computer program code for causing a computer to analyze a nucleotide sequence encompassing said genomic region of interest to identify repeat sequences within said nucleotide sequence;
computer program code for causing a computer to, for each subsequence of said nucleotide sequence that does not contain any of said repeat sequences, compare said subsequence against a nucleotide sequence database to identify nucleotide sequences within said database that are substantially similar to said subsequence;
computer program code for causing a computer to, for each of said subsequences for which a defined number of substantially similar sequences are found in said database, identify oligonucleotide sequences suitable for use as primers in an amplification reaction to amplify a product within said subsequence; and
computer program code for outputting said oligonucleotide sequences.

35. The method of claim 34, wherein said defined number of substantially similar sequences is zero.

36. The method of claim 34, wherein said substantially similar sequences are at least about 50% identical to said subsequences.

37. The method of claim 34, wherein said substantially similar sequences are at least about 70% identical to said subsequences.

38. The method of claim 34, wherein said substantially similar sequences are at least about 90% identical to said subsequences.

39. A method for identifying genes within a genomic region of interest, said method comprising the steps of:
executing a first process on a digital computer to identify repeat sequences that occur within said genomic region of interest;
executing a second process on a digital computer to compare repeat sequence-free subsequences within said genomic region of interest to a nucleotide sequence database, whereby nucleotide sequences within said nucleotide sequence database that are substantially similar to said repeat sequence-free subsequences are identified;
executing a third process on a digital computer to select repeat sequence-free subsequences having no substantially similar sequences to identify a repeat sequence-free subsequence may represent a gene family.
identify oligonucleotide sequences that are suitable for use as primers in an amplification reaction to amplify a product within any of said repeat sequence-free subsequences for which a defined number of substantially similar sequences are identified in said nucleotide sequence database; and
outputting said oligonucleotide sequences.
